(19) United States
(12) Patent Application Publication
(10) Pub. No.: US 2011/0195121 A1
(43) Pub. Date: Aug. 11, 2011

(54) CHRONOTHERAPEUTIC PHARMACEUTICAL DOSAGE FORM

(76) Inventors: Seshni Sewlall, Parkmore (ZA); Viness Pillay, Benmore (ZA); Yahya Choonara, Lenasia (ZA); Zaheeda Khan, Emmarentia (ZA)

(21) Appl. No.: 12/999,925

(22) PCT Filed: Jun. 3, 2009
(86) PCT No.: PCT/IB2009/005832

§ 371 Date: Apr. 15, 2011

(30) Foreign Application Priority Data
Jun. 19, 2008 (ZA) 122309

(51) Int. Cl. A61K 9/36; A61K 9/00; A61K 9/20; A61K 9/28; A61K 9/32; A61K 9/34; A61P 25/00; A61P 37/08

(52) U.S. Cl. 424/468; 424/400; 424/464; 424/475; 424/480; 424/482; 424/481

(57) ABSTRACT

This invention relates to a pharmaceutical dosage form for the phase-controlled and chronotherapeutic delivery of at least one and, preferably, several pharmaceutically active ingredients. The dosage form has a carrier platform which,—preferably, is a polymer having known biodegradable characteristics. The platform may include a pharmaceutically active ingredient'which is released over a predetermined period of time as the platform polymer degrades. At least one pharmaceutically active ingredient in the form of a disc is embedded in the platform and, once the polymer of the platform has degraded, the disc is released and releases its ingredient in the same location as that of the platform or it travels to another region of the body where it releases its ingredient.

### FIELD OF THE INVENTION

This invention relates to a pharmaceutical dosage form, more particularly; to a pharmaceutical dosage form suitable for the delivery of pharmaceutical compositions in a phase-controlled chronotherapeutic manner via the oral route or as an implantable embodiment in a human or animal body.

### BACKGROUND TO THE INVENTION

The treatment of certain disease states or disorders, commonly known as chronotherapeutic disorders, is compounded by the anomaly of circadian variations. This diurnal rhythm is synchronized by the sleep-wake pattern and manifests itself in various physiological processes in the body. Examples of chronotherapeutic disorders include respiratory diseases, cardiac diseases, rheumatoid arthritis, osteoarthritis and peptic ulcer disease. One example is cortisol secretion in the mammalian body which has been shown to be burst-like or pulsatile with a greater amplitude of release occurring in the early hours of the morning. Implications for cortisol release are seen in the treatments of adrenocorticoid insufficiency and other chronic inflammatory diseases such as rheumatoid arthritis and asthma.

In addition, clinical analyses of cardiovascular events like vasospastic angina pectoris, myocardial infarction and sudden cardiac death have displayed the effect of circadian rhythms in their increased tendencies to occur between night and early morning. Linked to the heart rate is the circadian pattern of blood pressure which normally rises and remains at its highest level for a few hours after awakening. Such events have given rise to the study of chronotherapy which assesses the effects of drug efficacy and clinical outcomes in a time-dependent manner.

The emphasis on the time of treatment rather than the type of treatment can therefore have many positive implications in controlling certain disorders and there are also suggestions that the pharmacokinetics and/or side effects of pharmaceutically active ingredients can be modified by the timing of their application within 24 hours of a day. Furthermore, diffusion of a pharmaceutically active ingredient should be modulated to release it in a time dependent manner in a 24 hour period, so that concentrations ideally fluctuate throughout the day.

In its simplest form, chronotherapy involves administering, to a patient in need thereof, pharmaceutically active ingredients at specific times of the day. While this is practical in a controlled environment such as a hospital of health care centre, it is not when the therapy is self administered, particularly when a treatment regime involved the administration of different pharmaceutically active ingredients at different times of the day. This is an obvious disadvantage.

The above difficulty has stimulated research into the development of alternative forms of orally administrable, modified release formulation pharmaceutical dosage forms which can provide staggered (e.g. biphasic and triphasic) release of a pharmaceutically active ingredient. These dosage forms, to a large extent, make use of technologies such as film-coating and compression-coating of cores containing a pharmaceutically active ingredient but there is a major disadvantage in that the rate of release of the pharmaceutically active ingredient tends to decrease towards the end of the release phase.

Numerous studies have been conducted on the use of multi-layered devices to address the above difficulties or disadvantages. Many of these focus on constant controlled drug release and not time controlled release (Whang et al. 2006, Conte et al., 1993, Georgiadis et a/0.2001, Martinez-Pacheco, 1986, Wan and Lai, 1992). Streubal et al. (2000) demonstrated that by using hydroxypropyl methylcellulose acetate succinate formulated in a multi-layered tablet, they were able to provide bimodal drug release (rapid release followed by constant release and a second phase of rapid release). However, in vivo the release of drug is strongly dependent on the gastric transit time as the second phase of drug release relies on a change in gastric pH. High variability in transit time leads to high variability of the time period separating the two rapid release phases. This device results in a quick onset of action, which may not necessarily be beneficial in chronotherapy.

Maggi et al. (1999) demonstrated that, by using a double-layered tablet, biphasic release could be achieved. The double-layered tablet comprised of one layer formulated to provide rapid drug release and the other released drug more slowly to maintain an effective plasma level for a prolonged period of time. This slow release was achieved by formulating the drug in a polymer matrix. This design, however, did not produce a lag time between the rapid drug release phase and the slow release phase.

In another study, Lopes et al. (2006) made use of mini-tablets compressed together to provide biphasic drug release. This device has an outer layer comprising of powder to provide rapid drug release, whereas the inner layer comprises the compressed mini-tablets and provides slower drug release. Here again, the design did not produce a lag time between the two phases of drug release.

U.S. Pat. No. 6,733,789 makes use of a multiparticulate bisoprolol formulation to treat hypertension using the concept of chronotherapy. The invention makes use of bisoprolol particles surrounded by a polymeric coating. This coating is able to provide an initial lag time of four to six hours after administration and is subsequently able to maintain a therapeutic concentration for a 24-hour period. The formulation is dosed every night such that there is a delay in drug release while the patient is asleep with release occurring prior to the patient wakening.

Mastiholimath et al, (2007), developed a hard gelatine capsule to release theophylline into the colon in a time and pH dependent manner in an attempt to treat nocturnal asthma. The entire capsule was coated in an enteric coating to prevent drug release in the stomach. In the intestine the enteric coating is released leaving behind a capsule within which is a swellable polymer. This prevents drug release in the small intestine and produces a lag phase. The drug is then released in the colon.

Even though both of these studies make use of chronotherapy to treat diseases, these designs provide an initial lag phase and then constant drug release.

### OBJECT OF THE INVENTION

It is an object of this invention to provide a pharmaceutical dosage form, more particularly a pharmaceutical dosage form which is suitable for the delivery of a pharmaceutical composition in a phase-controlled chronotherapeutic manner which, at least partly, alleviates the above mentioned disadvantages.

### SUMMARY OF THE INVENTION

In accordance with the invention there is provided a pharmaceutical dosage form for the phase-controlled and chronotherapeutic delivery of at least one pharmaceutically active ingredient, the pharmaceutical dosage form comprising a carrier composition platform and at least one pharmaceutically active ingredient which is at least partly embedded in the carrier composition platform, the carrier composition platform having predetermined degradation characteristics when in a human or animal body and, on degrading, in use, the pharmaceutically active ingredient is released in a phase-controlled and chronotherapeutic manner.

There is also provided for the pharmaceutically active ingredient to be in the form of a discrete pellet, preferably a disc, which is embedded in the platform and for the platform to be a polymer matrix of one or more polymers. Alternatively, there is provided for the pharmaceutically active ingredient to be mixed with the polymer or polymers forming the polymeric platform. Further alternatively there is provided for the pharmaceutically active ingredient to be pelletised and for the pellets to be embedded in the polymeric platform.

There is also provided for the pharmaceutical dosage form to include at least one and preferably, a plurality, of pellets containing at least one first pharmaceutically active ingredient within an operatively outer polymeric carrier composition coat, the operatively outer polymeric carrier composition coat having at least one second pharmaceutically active ingredient added thereto which is released, in a phase-controlled and chronotherapeutic manner when the operatively outer polymeric carrier composition coat degrades whereafter the pellet or pellets containing the first pharmaceutically active ingredient are released.

There is further provided for the first and second pharmaceutically active ingredients to be the same, alternatively different pharmaceutically active ingredients, for the first pharmaceutically active ingredient pellets to release the pharmaceutically active ingredient in the same or a different region of the human or animal body as that in which the second pharmaceutically active ingredient is released.

There is further provided for the pellets to be discoid, for the to be dimensioned and embedded within the operatively outer polymeric carrier composition coat so that, in use, the first and second pharmaceutically active ingredients are released over a desired period of time, preferably in a phase-controlled manner which may be rapid, alternatively slowly, as a result of variations in the diffusion pathlengths created.

There is further provided for the pellets to be coated with a pharmaceutical dosage form as claimed in any one of claims 34 to 37 in which a polymer, alternatively an enteric coating, for the coating to be polyvinyl acetate phthalate or cellulose acetate phalate, alternatively a specialized coating latex having a known dissolution rate of pH dependency so that, in use, the pharmaceutically active compound or compounds from either inner core tablet-like disc/s can be released over a desired period of time, preferably in a phase-controlled manner which may be rapid, alternatively slowly.

There is further provided for the pharmaceutically active compound contained within a multitude of inner core tablet-like discs embedded within the outer tablet-like platform to be granulated with a polymer, such as ethylcellulose or enteric coatings such as those from among the group comprising polyvinyl acetate phthalate, or a specialized coating latex having a known dissolution rate of pH dependency so that, in use, the pharmaceutically active compound or compounds from either inner core tablet-like disc/s can be released over a desired period of time, preferably in a phase-controlled manner which may be rapid alternatively slowly.

There is further provided for the polymeric platform to be formed from one or more polymers which may be a standard hydrophilic polymer, a hydrophillic swellable or erodible polymer, a standard hydrophobic polymer, a hydrophobic swellable/erodible polymer. Preferably the polymer is selected from the group consisting of: hydroxyethylcellulose (HEC), hydroxypropylcellulose (HPC), hydroxypropylmethylcellulose (HPMC), polyethylene oxide (PEO), polyvinyl alcohol (PVA), sodium alginate, pectin, ethylcellulose (EC), poly(lactic) co-glycolic acids (PLGA), polylactic acids (PLA), polymethacrylates, polycaprolactones, polyesters and polyamides, and for the polymer or polymers to be used alone or mixed with at least one co-polymer.

There is also provided for the dosage form to include a pharmaceutical excipient, preferably a lubricant such as magnesium stearate and/or a bulking agent such as lavtose and/or a crosslinking agent such as a salt.

There is also provided for the dosage form to include a superdisintegrant preferably sodium starch glycolate.

There is further provided for the dosage form components, particularly the polymers, to be selected so that, in use, there is an initial lag phase, a pharmaceutical active release phase and thereafter a second lag phase and further pharmaceutical active release, the above lag and release phases providing, in use, therapeutic blood levels similar to those produced by multiple smaller doses.

There is further provided for the said pharmaceutical dosage form to comprise embedded cores that may or may not be at an equal distance with respect to each other and the outer zones, a first outer zone, a middle zone and a second outer zone in which the symmetrically or asymmetrically embedded cores comprise one or more pharmaceutically active ingredients, the first outer zone partially surrounds one core, the second outer zone partially surrounds the other core, the middle zone separates at least two embedded cores and at least one of the first outer zone and the second outer zone comprises one or more pharmaceutically active ingredients, which one or more pharmaceutically active ingredients, are the same as or different than the one or more pharmaceutically active ingredients in the core, the first outer zone, the middle zone and the second outer zone are heterogeneous with respect to each other, the first outer zone and the second outer zone together form a continuous layer completely enclosing the cores, the first outer zone and the second outer zone together form a continuous layer completely enclosing the middle zone, the first outer zone comprises a barrier suitable for timed release of pharmaceutically active ingredients, the second outer zone comprises a barrier suitable for timed release of pharmaceutically active ingredients, the middle zone comprises a barrier suitable for timed release of pharmaceutically active ingredients and the cores, the first outer zone, the middle zone and the second outer zone together comprise a pharmaceutically effective dosage amount of each of the one or more pharmaceutically active ingredients.

There is also provided for the middle zone to incorporate a critical formulation excipient, preferably crosslinking reagents, solubilising agents, and/or other release-rate modulating composite polymers or polymer structures that is able to modulate the release of active pharmaceutical ingredient/s from pharmaceutically active ingredients embedded therein or encapsulated thereby.

### BRIEF DESCRIPTION OF THE FIGURES AND TABLES

The above and additional features of the invention will be described below by way of example only and with reference to the examples and to the accompanying Figures in which:

FIGS. 1A to J: Are schematic diagrams of various configurations of pharmaceutical dosage forms according to the invention;

FIG. 2: is a series of graphs of drug release profiles of multi-layered multi disc polymer (MLMDT) devices showing erratic drug release over 8 hours;

FIG. 3: is a series of graphs of drug release profiles of MLMDT devices showing controlled drug release with no lag phase;

FIG. 4: is a series of graphs of drug release profiles of MLMDT devices showing controlled drug release with a lag phase and up-curving release kinetics over 24 hours; and

FIG. 5: is a series of graphs of drug release profiles of MLMDT devices showing biphasic release over 120 hours.

FIG. 6: typical textural profiles for computing the physicomechanical properties of the MLMDT devices.

### DESCRIPTION OF THE PREFERRED EMBODIMENTS

Embodiments of the invention will be illustrated below by the following non-limiting examples.

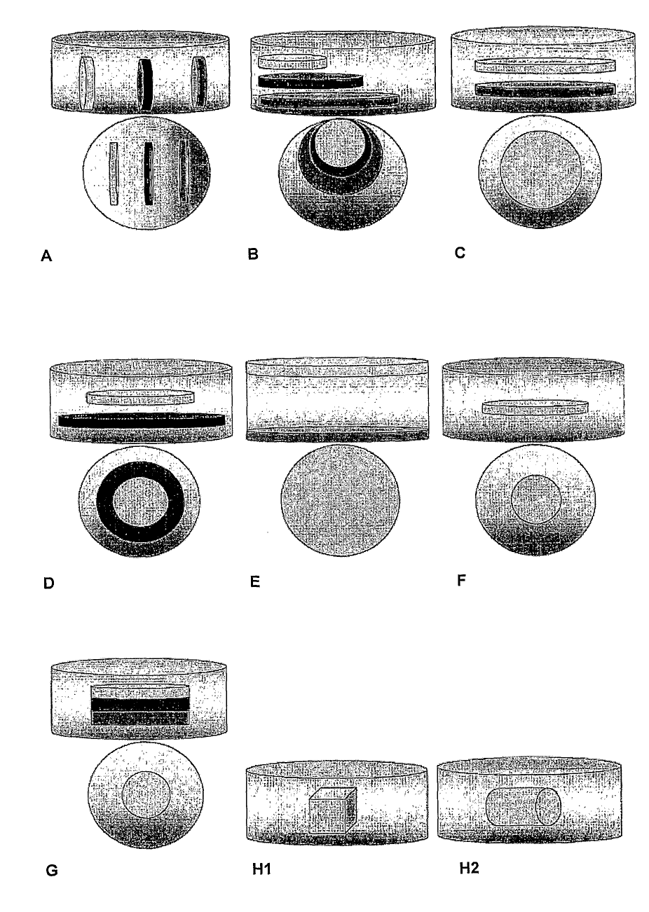

Referring to FIG. 1, a number of pharmaceutical dosage forms (1) in each in the form of an orally ingestible tablet are shown as FIGS. 1A to J as sectional side and plan views. Each dosage form (1) has a polymeric carrier composition platform (2) and at least one inclusion (3) containing a pharmaceutically active ingredient. The platform (2) has predetermined degradation characteristics when exposed to stimuli in the form of bodily secretions when ingested and, on degrading, release the pharmaceutically active ingredients (3) in a phase controlled and chronotherapeutic manner.

Referring specifically to FIG. 1A, the three inclusions (3) are in the form of similarly shaped and sized discs each containing a pharmaceutically active ingredient. The discs are embedded within the platform (2) and, when the platform degrades, the discs are freed and able to release the pharmaceutically active ingredient. The pharmaceutically active ingredient in each disc (3) may be coated with a coating composition which is, for example, resistant to degradation by gastric acids so that when the disc is freed it can pass through the stomach and into the small intestine or further to release its pharmaceutically active ingredient.

Referring specifically to FIG. 1B, the discs (3) are of different sizes and it is envisaged that this configuration can be used where substantially different doses of pharmaceutically active ingredients are to be delivered.

Referring to FIGS. 1C and 1D, these are substantially the same as those illustrated in FIGS. 1A and B except that only two discs are employed.

Referring to FIG. 1E, in this embodiment the discs are not embedded within the platform but are affixed to opposite sides of the tablet. It is envisaged that this configuration can be used where an immediate release of a pharmaceutically active ingredient is desired. In this embodiment the platform may, in the case of delivery to the stomach, be less dense than gastric juices and will float in the stomach until the platform has degraded.

Referring to FIG. 1F, here a single disc is employed but the platform may also contain a pharmaceutically active ingredient which is released as it degrades and, once degraded, the second ingredient in the disc is released in the same region of the body or in a different region.

Referring to FIG. 1G, here three discs are embedded in the platform and all three are released simultaneously once the platform degrades. In this case the platform may contain a pharmaceutically active ingredient for release in, for example, the stomach and, after its release the discs may migrate to another part of the gastrointestinal tract to release their ingredients or they may remain in the stomach.

Referring to FIGS. 1H1 to 1H4, alternative configurations to the discs as illustrated in the previous Figures are shown. In these embodiments the “discs” or pharmaceutically active ingredient inclusions are shaped to suit a particular rate of delivery of the pharmaceutically active ingredients.

FIGS. 1I and J also illustrate different configurations of the dosage form platforms.

Polymers suitable for oral dosage forms were identified based on available information provided in the literature. The compression properties of the various polymers (HPC, HEC and PEO) were assessed using a Beckman Hydraulic Press (Glenrothes, Scotland, UK). A punch and die set with a diameter of 10 mm was used at compression forces ranging from 5-10 tons. The compressibility of the polymer compacts were determined by the compression force which was represented by a conversion to the Brinell Hardness Number (BHN).

Polymers were selected for further manipulation based on their compressibility profiles. The devices were prepared through the use of customized pre-compression and final compression techniques and novel tooling developed in our laboratories. The upper and lower drug-loaded discs were separately compressed using a 5 mm flat-faced punch and die set in a Beckman Hydraulic Press (Beckman Instruments, Inc., Fullerton, USA). One of the discs was coated with an enteric coating using a Minilab® Fluid Bed Processor (DIOSNA, Osnabruck, Germany).

The influence of formulation variables such as polymer composition and concentration, and process variables such as compression pressure on the alteration of drug release and textural properties of the tablet device was elucidated through the application of statistical experimental design software. The preparation of the tablet device was repeated with the incorporation of electrolytes such as sodium carbonate and aluminium chloride into the drug-loaded discs and/or the polymeric layers in order to assess polymer-electrolyte interaction.

Drug release studies were performed in a six-station dissolution test apparatus (Caleva 7ST, Dorset, England) using a USP 29 Apparatus 2 in 900 mL USP-recommended buffers of pH 1.5, 4 and 6.8 at 37° C. and 50 rpm. Drug concentration was analyzed by ultraviolet spectroscopy (Specord 40, United Scientific, South Africa) at 280 nm for model drug theophylline and at 249 for model drug promethazine. Drug release studies were performed on the individually compressed drug-loaded layers as well as the final multi-layer multi-disc system.

To determine the effect of a continuous pH change with time, (i.e. simulated gastrointestinal pH variation), dissolution studies were also performed at 37±0.5° C. using a USP 29 Apparatus 3 (Bio-Dis II Release Rate Tester, Vankel Industries) at buffers of different pH (220 mL per vessel). Formulations were subjected in duplicate to a continuous run for 6 h each at pH 1.5 and 4, and 12 h at pH 6.8. The standard oscillation rate of 10 dpm was employed throughout the study. Samples were analyzed at time 0, 0.5, 2, 4, 6, 10, 12, 18, 24 hours and results analyzed by Ultra Performance Liquid Chromatography (HPLC).

Variations in the physicomechanical properties of the compressed tablet devices were assessed using a Texture Analyzer (TA.XT plus, Stable Microsystems, UK). Samples were immersed in 900 mL buffer medium (pH 1.5, 3 and 6.8; 37° C.) with paddle speed set at 50 rpm in a dissolution apparatus. At pre-determined time intervals, samples (N=10) were removed and subjected to Force-Distance and Force-Time profiling using a flat-tipped 2 mm cylindrical steel probe.

Tablet configurations with and without electrolytes were hydrated in buffer media of pH 1.5, 3 and 6.8. At pre-determined time intervals, samples were removed (N=10) and characterized by darkfield stereomicroscopy (SZX7, Olympus Corporation, Tokyo, Japan) in order to view the changes in peripheral and glassy core regions. Analysis Starter® software (Version 3.2, Soft Imaging System, Germany) was used to make measurements at the micrometer level to ensure accuracy.

A one-way Analysis of Variance (ANOVA) was conducted on each of the responses (i.e. dependent variables) at a 95% confidence interval in order to determine the level of interaction among the independent variables (main effects). Since a three-level full factorial design was used, the following indices were monitored: R², Durbin-Watson Statistic and PRESS Index to ensure model suitability and stability. Whenever possible, the experimental optimization technique of factorial design was utilized. Release data was modeled using pharmacokinetic software namely, WinNonLin Version 5.1 (Pharsight software, USA.).

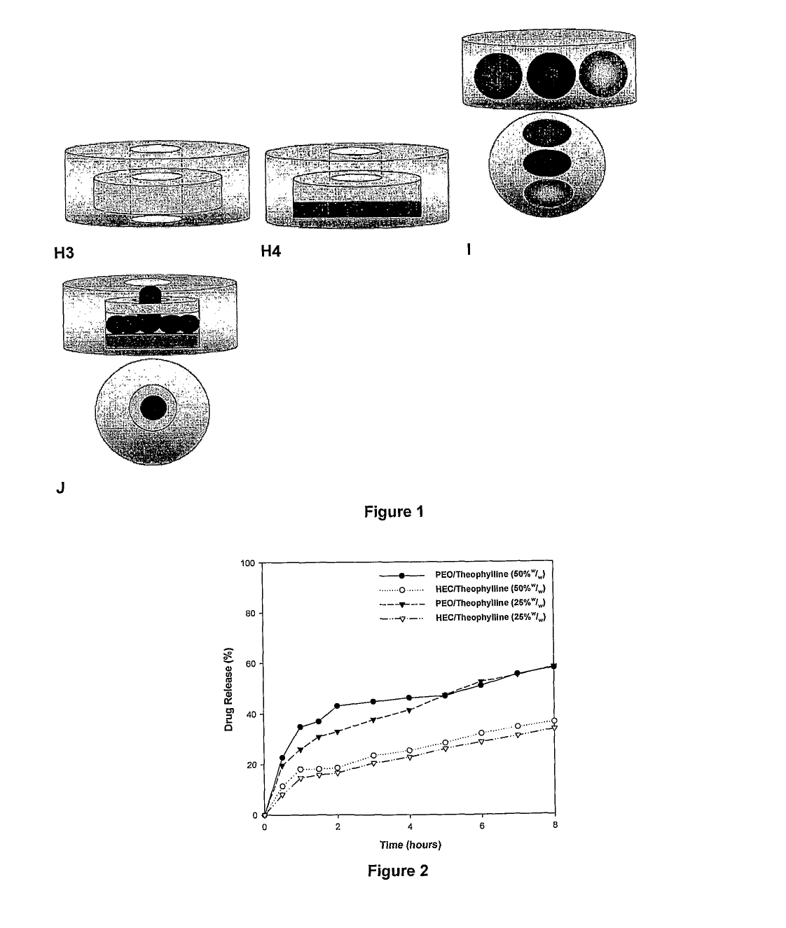

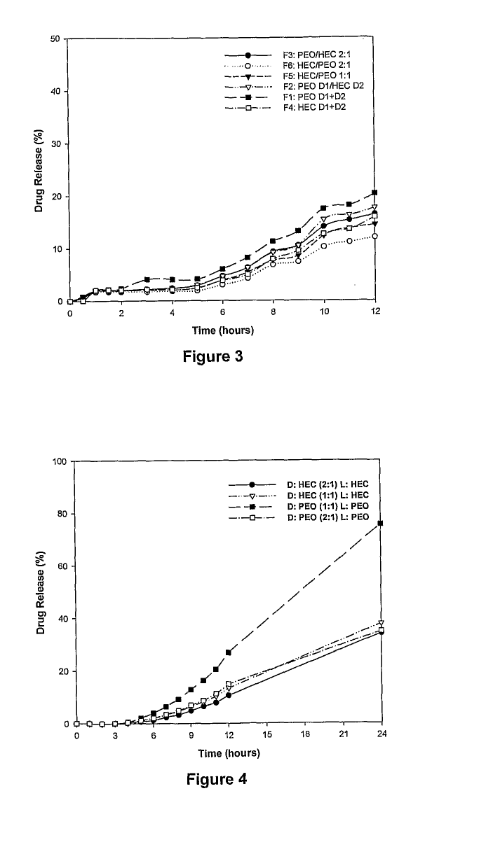

Initial ratios and combinations of discs suspended within hydroxyethylcellulose (HEC) layers showed erratic and unpredictable drug release profiles (FIG. 2). The introduction of polyethylene oxide (PEO) into the outer layers (FIGS. 3, and 4) provided more stable and regulated drug release, with an initial lag phase and a potential for biphasic release. However, drug release at the 24-hour time interval did not exceed 31%. A subsequent study using similar dimensions with only polyethylene oxide (PEO) in the outer layers displayed drug release of 70-90% at the 48-hour time interval. In order to reduce the profile to 24 hours to achieve the ideal therapeutic period for chronotherapy, the concentration of polymer in the outer layers was decreased and resulted in increased drug release (FIG. 4) at the 24-hour time interval (50-80%).

The next step was to concentrate on the drug-loaded discs. The ratios of polymer to drug were varied in order to induce a change in the release rate from the discs. This resulted in pseudo zero-order/slow-upcurving kinetics (FIG. 4) with a drug release of 80-100% at 24 hours. The lack of a significant initial lag phase led to a further study (FIG. 3) in which the concentration of polymer surrounding the discs was increased and the ratio of drug in the two discs varied. However, it became evident that while an increased concentration of polymer in the outer layers induced an initial lag phase, it was at the expense of decreasing the drug release rate to extend beyond the 24-hour time interval.

FIG. 2 depicts the erratic release patterns achieved with conventional HEC and PEO matrices. Drug release profiles with an initial lag phase and slow up-curving kinetics were achieved employing PEO in the outer layers and HEC in the disc layers (FIG. 3). A change in the ratio and/or concentration of polymer resulted in similar release profiles with ranges of 50-80%, 70-90% and 80-100% drug release at the 24 hour time interval (FIG. 4). A correlation between the concentration of polymer, lag phase induction and % drug release was noted.

Robust matrices were produced upon compression of HEC, PEO and the drug-loaded discs (Table 1).

TABLE 1
Compressibility of each polymer grade
Table 1. Compressibility of each polymer grade

| Polymer type | [% ^{w}/_{w}] | Force (tons) | ^{a}BHN (N/mm²) |
|---|---|---|---|
| PEO | 500 mg | 8 tons | 5.896 |
| HEC | 500 mg | 8 tons | 4.141 |
| HPC | 500 mg | 8 tons | 2.391 |

^{a}= Brinell hardness number

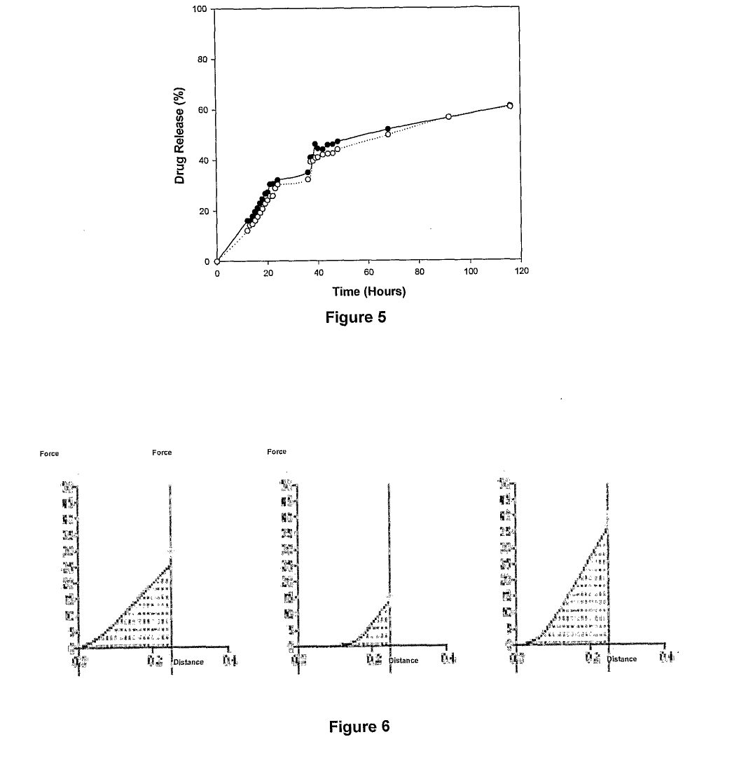

Textural analysis confirmed Brinell Hardness Number (BHN) values to range from 2.071-2.949 N/mm² which demonstrated desirable compressibility characteristics (FIG. 6). HEC and PEO were used as a retentive mechanism in achieving a significant lag phase of between 3-5 hours prior to drug release. Drug release occurred in a phasic release pattern with an initial lag-phase and a subsequent exponential release phase to completion. This biphasic release ranged from 7-26% at t₁₂ₕₒᵤᵣₛ followed by 19-75% at t₂₄ₕₒᵤᵣₛ (FIG. 5).

This work has resulted in the successful design of a multi-layered multi-disc device for phase-controlled chronotherapeutic drug delivery. In vitro studies have shown the potential for desirable drug release kinetics. These studies have also exhausted the possibilities of combinations between the polymers used, which led to further studies where different polymers/electrolytes/other materials were introduced into the outer layers to control drug release from the discs. An ideal formulation was achieved and optimized with the use of a statistical design and further textural profiling, polymer viscosity, erosion/swelling and HPLC studies were conducted. The multi-layered multi-disc polymeric device was successfully designed for phase-controlled drug delivery, which demonstrates desirable release kinetics for chronotherapeutic disorders.

1. A pharmaceutical dosage form for the phase-controlled and chronotherapeutic delivery of at least one pharmaceutically active ingredient, the pharmaceutical dosage form comprising a carrier composition platform and at least one pharmaceutically active ingredient which is at least partly embedded in the carrier composition platform, the carrier composition platform having predetermined degradation characteristics when in a human or animal body and, on degrading, in use, the pharmaceutically active ingredient is released in a phase-controlled and chronotherapeutic manner.

2. A pharmaceutical dosage form as claimed in claim 1 in which the pharmaceutically active ingredient to be in the form of a discrete pellet which is embedded in the platform.

3. A pharmaceutical dosage form as claimed in claim 2 in which discrete pellet is in the form of a disc.

4. A pharmaceutical dosage form as claimed in claim 1 in which the pharmaceutically active ingredient is mixed with the polymer or polymers forming the polymeric platform.

5. A pharmaceutical dosage form as claimed in claim 1 in which the pharmaceutically active ingredient is pelletised and the pellets are embedded in a polymeric platform.

6. A pharmaceutical dosage form as claimed in claim 1 in which at least one or a plurality, of pellets containing at least one first pharmaceutically active ingredient is located within an operatively outer polymeric carrier composition coat which has at least one second pharmaceutically active ingredient added thereto which is released, in a phase-controlled and chronotherapeutic manner when the operatively outer polymeric carrier composition coat degrades whereafter the pellet or pellets containing the first pharmaceutically active ingredient is released.

7. A pharmaceutical dosage form as claimed in claim 1 in which a plurality of pellets containing at least one first pharmaceutically active ingredient are located within an operatively outer polymeric carrier composition coat which has at least one second pharmaceutically active ingredient added thereto which is released, in a phase-controlled and chronotherapeutic manner when the operatively outer polymeric carrier composition coat degrades whereafter pellets containing the first pharmaceutically active ingredient are released.

8. A pharmaceutical dosage form as claimed in claim 6 in which the first and second pharmaceutically active ingredients are the same.

9. A pharmaceutical dosage form as claimed in claim 6 in which the first and second pharmaceutically active ingredients are different pharmaceutically active ingredients.

10. A pharmaceutical dosage form as claimed in claim 6 in which the first pharmaceutically active ingredient pellets release the pharmaceutically active ingredient in the same or a different region of the human or animal body as that in which the second pharmaceutically active ingredient is released.

11. A pharmaceutical dosage form as claimed in claim 5 in which the pellets are discoid.

12. A pharmaceutical dosage form as claimed in claim 11 in which the pellets are embedded within an operatively outer polymeric carrier composition coat so that, in use, a first and second pharmaceutically active ingredients are released over a desired period of time which may be rapidly or slowly, the rate of release being a function of variations in the diffusion pathlengths created.

13. A pharmaceutical dosage form as claimed in claim 12 in which the first and second pharmaceutically active ingredients are released in a phase-controlled manner.

14. A pharmaceutical dosage form as claimed in claim 5 in which the pellets are coated with a polymer.

15. A pharmaceutical dosage form as claimed in claim 5 in which the pellets are coated with an enteric coating.

16. A pharmaceutical dosage form as claimed in claim 14 in which the enteric coating is polyvinyl acetate phthalate or cellulose acetate phalate.

17. A pharmaceutical dosage form as claimed in claim 14 in which the enteric coating is a specialized coating latex having a known dissolution rate which is pH dependent so that, in use, the pharmaceutically active compound or compounds are released over a desired period of time.

18. A pharmaceutical dosage form as claimed in claim 17 in which the pharmaceutically active compound or compounds are released in a phase-controlled manner which may be rapid or slowly.

19. A pharmaceutical dosage form as claimed in claim 1 in which the pharmaceutically active compound is contained in a plurality of inner core tablet-like discs which are embedded within an outer tablet-like platform.

20. A pharmaceutical dosage form as claimed in claim 19 in which the pharmaceutically active compound is granulated with a polymer or with an enteric coating.

21. A pharmaceutical dosage form as claimed in claim 20 in which the polymer is ethylcellulose.

22. A pharmaceutical dosage form as claimed in claim 20 in which the enteric coating is polyvinyl acetate phthalate or a specialized coating latex having a known dissolution rate of pH dependency so that, in use, the pharmaceutically active compound or compounds from either inner core tablet-like disc/s can be released over a desired period of time.

23. A pharmaceutical dosage form as claimed in claim 21 in which the pharmaceutically active compound or compounds is or are released in a phase-controlled manner which may be rapidly or slowly.

24. A pharmaceutical dosage form as claimed in claim 4 in which the polymeric platform is formed from one or more polymers which is: a standard hydrophilic polymer, a hydrophilllic swellable or erodible polymer, a standard hydrophobic polymer, or a hydrophobic swellable/erodible polymer.

25. A pharmaceutical dosage form as claimed in claim 24 in which the polymer is selected from the group consisting of: hydroxyethylcellulose (HEC), hydroxypropylcellulose (HPC), hydroxypropylmethylcellulose (HPMC), polyethylene oxide (PEO), polyvinyl alcohol (PVA), sodium alginate, pectin, ethylcellulose (EC), poly(lactic) co-glycolic acids (PLGA), poly lactic acids (PLA), polymethacrylates, polycaprolactones, polyesters and polyamides.

26. A pharmaceutical dosage form as claimed in claim 24 in which the polymer or polymers are used alone or are mixed with at least one co-polymer.

27. A pharmaceutical dosage form as claimed in claim 1 in which the dosage form includes at least one pharmaceutical excipient.

28. A pharmaceutical dosage form as claimed in claim 27 in which the pharmaceutical excipient is a lubricant.

29. A pharmaceutical dosage form as claimed in claim 27 in which the pharmaceutical excipient is a bulking agent.

30. A pharmaceutical dosage form as claimed in claim 27 in which the pharmaceutical excipient is a crosslinking agent.

31. A pharmaceutical dosage form as claimed in claim 27 in which the dosage form includes a superdisintegrant.

32. A pharmaceutical dosage form as claimed in claim 1 in which the dosage form components are selected so that, in use, there is an initial lag phase, a pharmaceutical active release phase and thereafter a second lag phase and further pharmaceutical active release.

33. A pharmaceutical dosage form as claimed in claim 32 in which the lag and release phases provided, in use, therapeutic blood levels similar to those produced by multiple smaller doses.

34. A pharmaceutical dosage form as claimed in claim 1 in which the said pharmaceutical dosage form has at least one central embedded core or a plurality of embedded cores, each core having an operatively first outer zone, and an operatively second outer zone with the central cores including one or more pharmaceutically active ingredients.

35. A pharmaceutical dosage form as claimed in claim 34 in which the dosage form has a plurality of embedded cores which are equidistantly spaced apart from each other.

36. A pharmaceutical dosage form as claimed in claim 34 in which the dosage form has a plurality of embedded cores which are not equidistantly spaced apart from each other.

37. A pharmaceutical dosage form as claimed in claim 34 in which the first operatively outer zone at least partially surrounds one core and in which the second operatively outer zone at least partially surrounds the other core.

38. A pharmaceutical dosage form as claimed in claim 35 in which, in addition to the first outer zone and the second outer zone, the dosage form has a middle zone in which the cores are embedded.

39. A pharmaceutical dosage form as claimed in claim 34 in which at least one of the first outer zone and the second outer zone includes one or more pharmaceutically active ingredients.

40. A pharmaceutical dosage form as claimed in claim 39 in which both zones contain one or more pharmaceutically active ingredients which are the same as or different to the one or more pharmaceutically active ingredients in the core or cores.

41. A pharmaceutical dosage form as claimed in claim 38 in which the middle zone also contains one or more pharmaceutically active ingredients which are the same as or different to the one or more pharmaceutically active ingredients in the outer zones.

42. A pharmaceutical dosage form as claimed in claim 38 in which the middle zone is completely encapsulated by the first and/or second outer zones.

43. A pharmaceutical dosage form as claimed in claim 34 in which the first operatively outer zone and/or the second operatively outer zone and/or the middle zone are heterogeneous with respect to each other.

44. A pharmaceutical dosage form as claimed in claim 34 in which, the first operatively outer zone and the second operatively outer zone together form a continuous layer completely enclosing the cores.

45. A pharmaceutical dosage form as claimed in claim 34 in which, each zone includes a barrier suitable for timed release of pharmaceutical active ingredients contained therein or encapsulated thereby.

46. A pharmaceutical dosage form as claimed in claim 34 in which, the cores, the first operatively outer zone, the second operatively outer zone, and the middle zone, together, comprise a pharmaceutically effective dosage amount of each of the one or more pharmaceutically active ingredients.

47. A pharmaceutical dosage form as claimed in any one of claim 38 in which the middle zone incorporates a critical formulation excipient, that is able to modulate the release of active pharmaceutical ingredient/s from pharmaceutically active ingredients embedded therein or encapsulated thereby.

48. A pharmaceutical dosage form as claimed in claim 27 in which the pharmaceutical excipient is selected from a lubricant, a bulking agent and a crosslinking agent.

49. A pharmaceutical dosage form as claimed in claim 28 in which the lubricant is magnesium stearate.

50. A pharmaceutical dosage form as claimed in claim 29 in which the bulking agent is lactose.

51. A pharmaceutical dosage form as claimed in claim 30 in which the crosslinking agent is a salt.

52. A pharmaceutical dosage form as claimed in claim 36 in which, in addition to the first outer zone and the second outer zone, the dosage form has a middle zone in which the cores are embedded.

53. A pharmaceutical dosage form as claimed in claim 52 in which the middle zone also contains one or more pharmaceutically active ingredients which are the same as or different to the one or more pharmaceutically active ingredients in the outer zones.

54. A pharmaceutical dosage form as claimed in claim 52 in which the middle zone is completely encapsulated by the first and/or second outer zones.

55. A pharmaceutical dosage form as claimed in claim 52 in which the middle zone incorporates a critical formulation excipient that is able to modulate the release of active pharmaceutical ingredient/s from pharmaceutically active ingredients embedded therein or encapsulated thereby.

56. A pharmaceutical dosage form as claimed in any one of claim 47 in which the critical formulation excipient is selected from: crosslinking reagents, solubilising agents, release-rate modulating composite polymers and polymer structures.

57. A pharmaceutical dosage form as claimed in any one of claim 55 in which the critical formulation excipient is selected from: crosslinking reagents, solubilising agents, release-rate modulating composite polymers and polymer structures.
